(19) United States
(12) Patent Application Publication
(10) Pub. No.: US 2007/0295916 A1
(43) Pub. Date: Dec. 27, 2007

(54) Toothbrush Head Sanitizing Cap with Built-In Ultraviolet LED.

(76) Inventor: David Isidore Reuben, Las Vegas, NV (US)

Correspondence Address: DAVID REUBEN, 10708 MOON FLOWER ARBOR PLACE, LAS VEGAS, NV, 89144, US

(21) Appl. No.: 11/426,066

(22) Filed: Jun. 23, 2006

(51) Int. Cl. A61L 2/10

(52) U.S. Cl. 250/455.11

(57) ABSTRACT

A toothbrush cap that contains an ultraviolet LED and slips over the head of a toothbrush in order to sanitize it. In the preferred embodiment the built-in LED and battery circuit is designed to have a lifetime that coincides with the lifetime of the toothbrush. In the preferred embodiment the cap is disposed of along with the toothbrush at the end of their lifetime or the cap is sent in for recycling.

### BACKGROUND OF THE INVENTION

The Toothbrush Head Sanitizing Cap with Built-In Ultraviolet LED is intended to be a mass consumer product. The engineering of the cap combines the fields of science of photobiology and optoelectronics with electrical circuit design.

Ultraviolet germicidal irradiation (UVGI) is known to be an effective method of killing a broad range of microbes. Wavelengths at or near 2537 Angstroms are uniquely destructive to microbes due to the fact that it corresponds to the structural resonance of DNA for micro-organisms. In this toothbrush cap the source of the UVGI is an ultraviolet LED.

UVGI inactivates pathogens according to the standard decay equation S=EXP(−klt) where S represents the fraction of the original pathogen population that survives exposure time t, and l represents the UVGI intensity. The rate constant k has been determined experimentally for a number of bacteria, viruses and spores, at different power levels.

In the case of the toothbrush cap the time t would be the time between brushing and the desired S would be determined by the efficacy requirement for marketing the product.

The optical radiation emitted by the LED is strongly correlated to the electrical current. And the electrical current correlates to the lifetime of the battery which powers it according to the equation life(hours)=(ampere-hour rating)/(amperes drawn).

Design criteria for the circuit involve determination of current requirements to reach desired UVGI intensity that meet efficacy requirements. Commercial availability and cost of the battery and LED are also considerations in the final selection of the components.

Continuous advancement in LED and battery technology are working to increase the efficacy and decrease the cost of this product ensuring its commercial viability.

### REFERENCES

1. Penn State Department of Architectural Engineering (2006). “Ultraviolet Irradiation”. Web.

2. Labsphere.com (2006). “The Radiometry of Light Emitting Diodes”. Web.

### DRAWINGS—FIGURES

FIG. 1 shows side view of cap

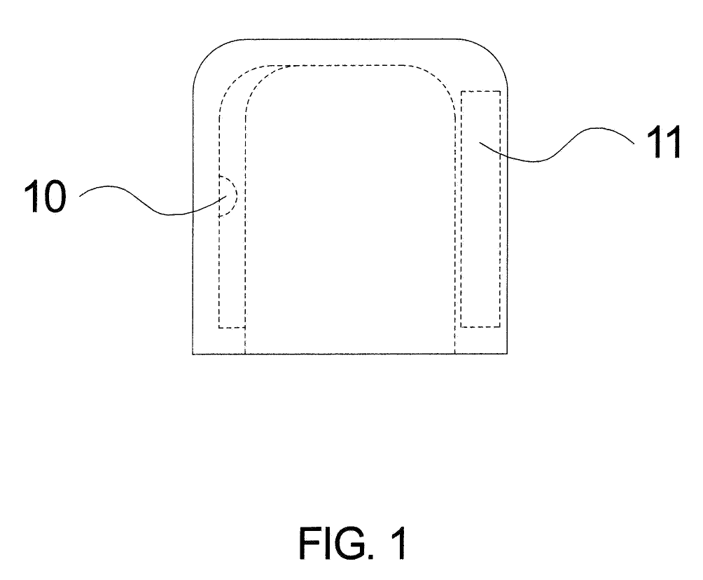

FIG. 2 shows front view of cap

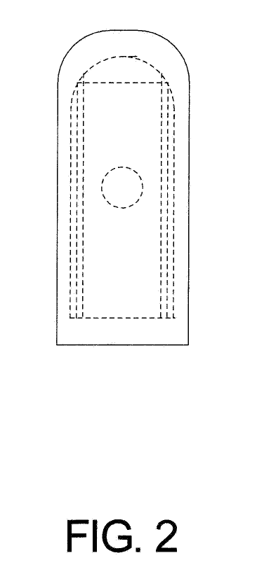

FIG. 3 shows side view of cap on toothbrush

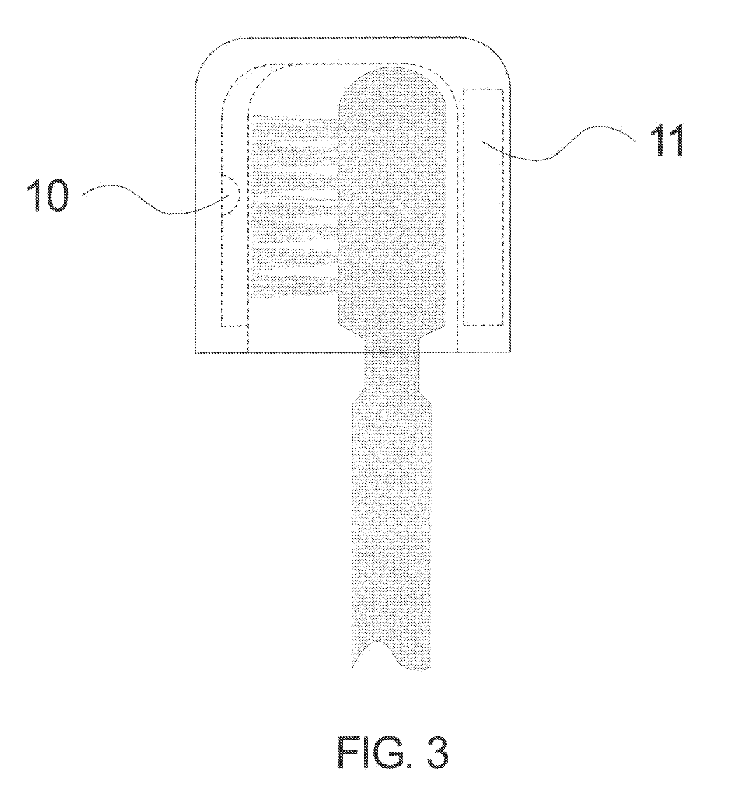

FIG. 4 shows the electrical schematic

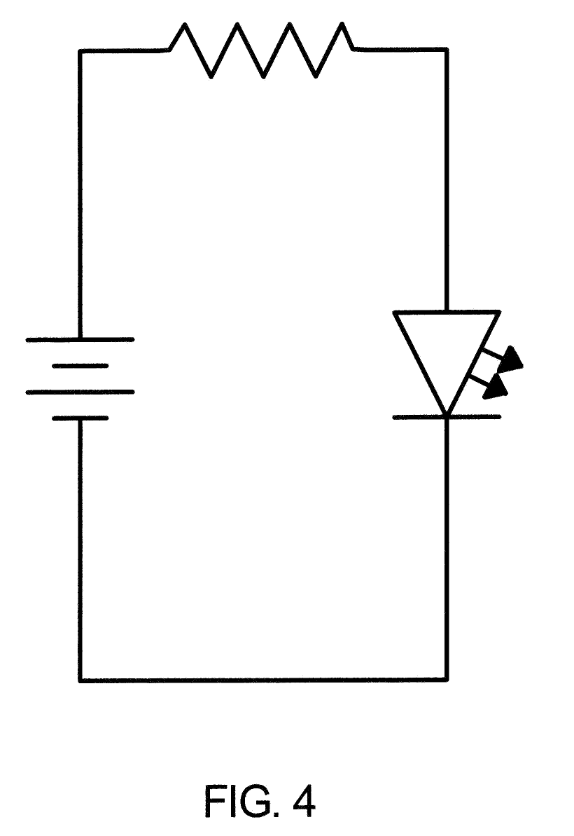

### DRAWINGS—REFERENCE NUMERALS

10 Ultraviolet LED

11 Battery

I claim:

1. A toothbrush head cap containing one or more ultraviolet LED's and a DC power source.

2. The DC power source in claim one is permanently embedded in the cap.

3. The DC power source in claim one is a replaceable battery.

4. The DC power source in claim one is a solar cell.

5. The LED or LED's in claim one stay on continuously.

6. The LED or LED's in claim one are controlled by a timer.

7. The LED or LED's in claim one are pulsed.

8. The LED or LED's in claim one illuminate the toothbrush head directly.

9. The LED or LED's in claim one illuminate the toothbrush head through a translucent pane or plurality of translucent panes.

10. The LED or LED's in claim one illuminate the toothbrush head through a filter.

11. The ultraviolet LED or LED's and DC power source in claim one are on the same side of the cap.

12. The ultraviolet LED or LED's and DC power source in claim one are on different sides of the cap.

13. The toothbrush cap in claim one slips onto the toothbrush head horizontally.

14. The toothbrush cap in claim one slips onto the toothbrush head vertically.
